(19) United States
(12) Patent Application Publication
(10) Pub. No.: US 2012/0004530 A1
(43) Pub. Date: Jan. 5, 2012

(54) QUANTIFICATION OF INTRACELLULAR AND EXTRACELLULAR SPIO AGENTS WITH R2 AND R2* MAPPING

(75) Inventors: Wei Liu, Rockville, MD (US); Julien Senegas, Hamburg (DE); Stefanie Remmele, Hamburg (DE)

(73) Assignee: KONINKLIJKE PHILIPS ELECTRONICS N.V., EINDHOVEN (NL)

(21) Appl. No.: 13/256,485

(22) PCT Filed: Feb. 9, 2010
(86) PCT No.: PCT/IB2010/050586

§ 371 Date: Sep. 14, 2011

Related U.S. Application Data
(60) Provisional application No. 61/163,062, filed on Mar. 25, 2009.

(51) Int. Cl. A61B 5/055; A61K 49/08

(52) U.S. Cl. 600/410; 424/9.36

(57) ABSTRACT

Quantitative assessment of magnetic agent tagged cells in a subject comprises: acquiring a series of T2 weighted images of the subject; acquiring a series of T2* weighted images of the subject; and generating a value indicative of quantitative assessment of magnetic agent tagged cells in the subject based on both the T2 weighted images of the subject and the T2* weighted images of the subject. The generating may be further based on predetermined relationships (26) between (i) R2 and intracellular magnetic agent concentration, (ii) R2* and intracellular magnetic agent concentration, (iii) R2 and extracellular magnetic agent concentration, and (iv) R2* and extracellular magnetic agent concentration. Said predetermined relationships may be generated based on R2 and R2* measurements of a plurality of calibration phantoms having different concentrations of substantially purely intracellular magnetic agent and having different concentrations of substantially purely extracellular magnetic agent.

The following relates to the medical arts, magnetic resonance arts, and related arts.

Interventional techniques, such as stem cell therapies, which entail administering biological cells to a subject are naturally sensitive to the distribution of cells in the subject. A known method for assessing the distribution of cells in the subject is to tag the cells with a magnetic agent, such as a superparamagnetic iron oxide (SPIO) agent, and to image the subject using magnetic resonance (MR) imaging. In a typical stem cell therapy approach, the stem cells are cultured in a medium containing an SPIO agent. After culturing, the cells are processed to remove the extracellular SPIO agent and then are administered to the subject. In the subject, the SPIO agent disrupts the magnetic field in the vicinity of the SPIO-tagged cells, which reduces the magnetic resonance spin relaxation time. A T2 or T2* weighted image (or, equivalently, a R2 or R2* image where R2=1/T2 and R2*=1/T2*) thus provides contrast for the SPIO-tagged cells.

This technique has been shown to be qualitatively effective. However, attempts to quantify the density of SPIO-tagged cells have been less successful. It is known that the T2 and T2* signals are differently affected by intracellular SPIO as compared with extracellular SPIO. This has led to speculation that incomplete removal of the extracellular SPIO or release of SPIO to extracellular space after cell death may be preventing reliable quantification of the SPIO-tagged cell concentration, although other factors such as hemorrhaging, cell necrosis, cell morphology and charging effects, and so forth have also been cited as possible causes. See Kuhlpeter et al., “R2 and R2* Mapping for Sensing Cell-bound Superparamagnetic Nanoparticles: In Vitro and Murine in Vivo Testing”, Radiology vol. 245 no. 2, pp. 449-57 (2007); Rad et al., “Quantification of Superparamagnetic Iron Oxide (SPIO)-Labeled Cells Using MRI”, Journal of Magnetic Resonance Imaging vol. 26 pp. 366-74 (2007).

In accordance with certain illustrative embodiments shown and described as examples herein, a method is disclosed for quantitative assessment of magnetic agent tagged cells in a subject, the method comprising: acquiring a series of T2 weighted images of the subject; acquiring a series of T2* weighted images of the subject; and generating a value indicative of quantitative assessment of magnetic agent tagged cells in the subject based on both the T2 weighted images of the subject and the T2* weighted images of the subject.

In accordance with certain additional illustrative embodiments shown and described as examples herein, a magnetic resonance imaging system configured to perform a method as set forth in the immediately preceding paragraph is disclosed, and a digital storage medium storing instructions executable to cause a magnetic resonance imaging system to perform a method as set forth in the immediately preceding paragraph is disclosed. The digital storage medium may, for example, be a magnetic disk, an optical disk, an electrostatic memory, a random access memory (RAM), a read-only memory (ROM), or so forth.

In accordance with certain illustrative embodiments shown and described as examples herein, a system is disclosed for quantitative assessment of magnetic agent tagged cells in a subject, the system comprising: a magnetic resonance imaging system; and a processor configured to cause the magnetic resonance imaging system to acquire both T2 weighted and T2* weighted images of the subject and further configured to generate a value indicative of quantitative assessment of magnetic agent tagged cells in the subject based on both the T2 weighted and T2* weighted images

One advantage resides in more accurate assessment of the distribution or density of magnetic agent-tagged cells using MR imaging.

Another advantage resides in improved assessment of interventional techniques, such as stem cell therapies, which entail administering biological cells to a subject.

Further advantages will be appreciated to those of ordinary skill in the art upon reading and understand the following detailed description.

The drawings are only for purposes of illustrating the preferred embodiments, and are not to be construed as limiting the invention.

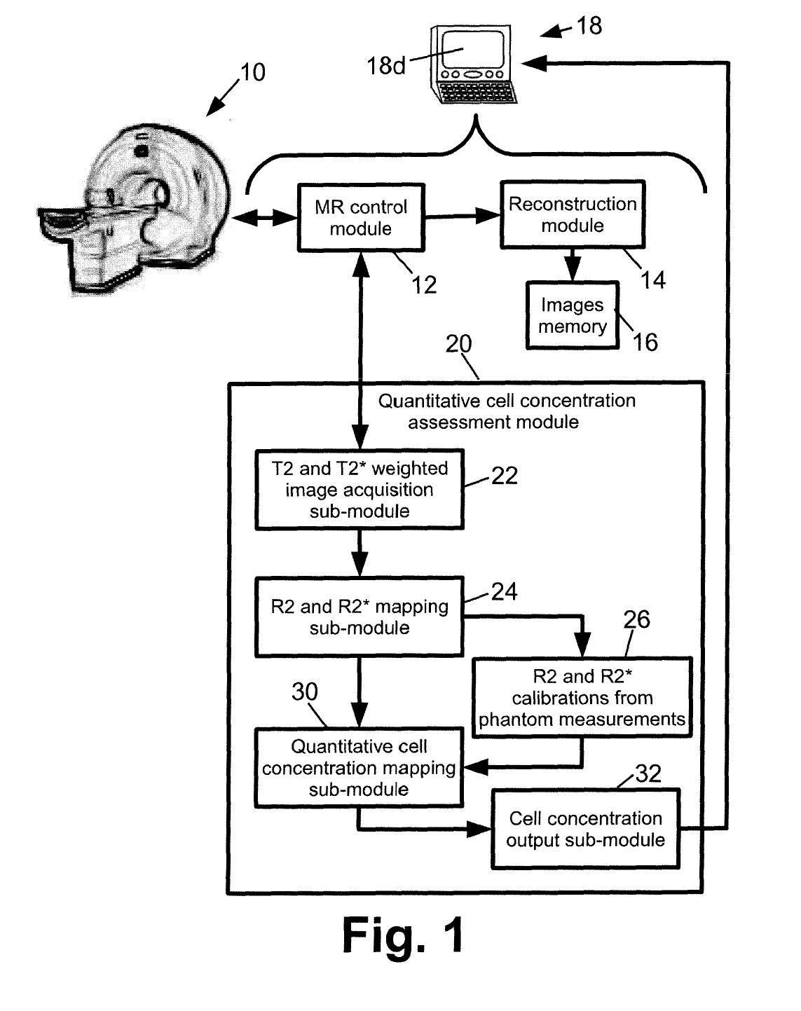

FIG. 1 diagrammatically shows a system for quantitative assessment of magnetically tagged cell concentrations using magnetic resonance imaging.

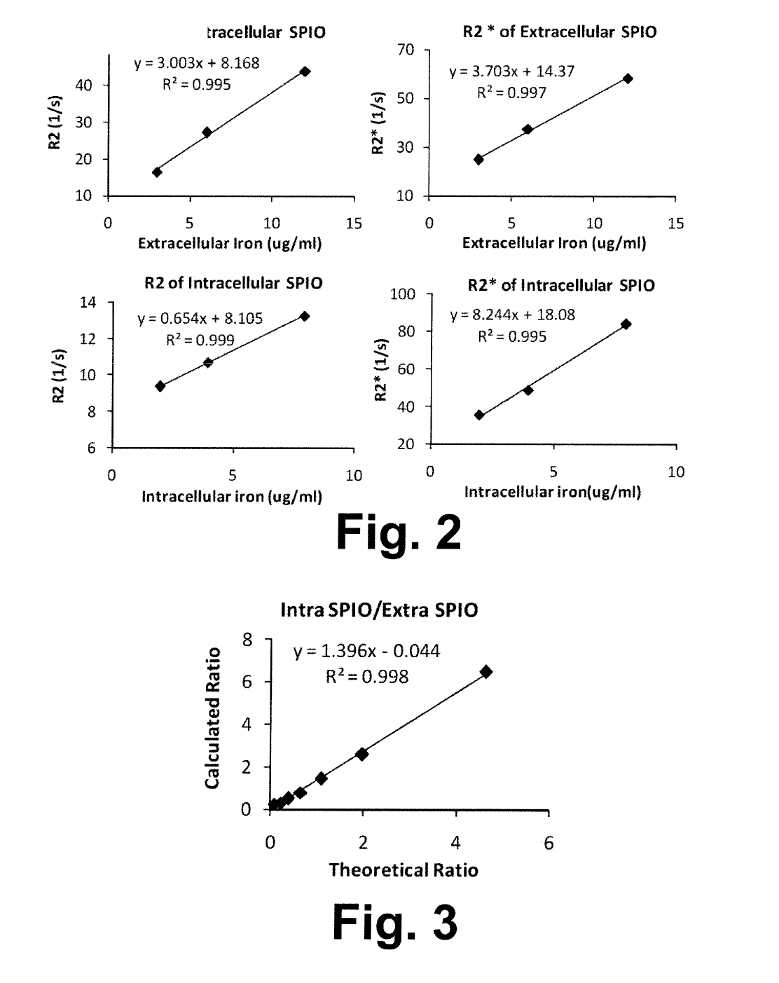

FIG. 2 diagrammatically shows calibration data for use in the system of FIG. 1 acquired from phantoms.

FIG. 3 diagrammatically shows estimated ratios of the intracellular and extracellular SPIOs as compared with theoretical values for these ratios.

With reference to FIG. 1, a magnetic resonance (MR) imaging system includes a magnetic resonance scanner 10, such as an illustrated Achieva™ magnetic resonance scanner (available from Koninklijke Philips Electronics N.V., Eindhoven, The Netherlands), or an Intera™ or Panorama™ magnetic resonance scanner (both also available from Koninklijke Philips Electronics N.V.), or another commercially available magnetic resonance scanner, or a non-commercial magnetic resonance scanner, or so forth. In a typical embodiment, the magnetic resonance scanner includes internal components (not illustrated) such as a superconducting or resistive main magnet generating a static (B₀) magnetic field, sets of magnetic field gradient coil windings for superimposing selected magnetic field gradients on the static magnetic field, a radio frequency excitation system for generating a radiofrequency (B₁) field at a frequency selected to excite magnetic resonance (typically ¹H magnetic resonance, although excitation of another magnetic resonance nuclei contained in the placenta is also contemplated), and a radio frequency receive system including a radio frequency receive coil, or an array of two, three, four, eight, sixteen, or more radio frequency receive coils, for detecting magnetic resonance signals emitted from the subject.

The magnetic resonance scanner 10 is controlled by a magnetic resonance control module 12 to execute a magnetic resonance imaging scan sequence that defines the magnetic resonance excitation, spatial encoding typically generated by magnetic field gradients, and magnetic resonance signal readout. A reconstruction module 14 reconstructs acquired magnetic resonance signals to generate magnetic resonance images or spatial maps that are stored in a magnetic resonance images memory 16. In some embodiments, the components 12, 14, 16 are general-purpose commercial magnetic resonance imaging products provided by the manufacturer of the magnetic resonance scanner 10 and/or by one or more third party vendors, for example embodied as software executing on a digital processor (not shown) of an illustrated computer 18. Alternatively, one or more or all of the components 12, 14, 16 may be custom-built or customer-modified components.

A quantitative cell concentration assessment module 20 configures the magnetic resonance imaging system to perform quantitative assessment of tagged cell concentrations, or distributions of such concentrations, in a subject. The module 20 may for example be embodied as software executing on a digital processor of the illustrated computer 18, or may be embodied as an interacting separate digital processor.

Heretofore, the washing or other processing to remove the extracellular SPIO or other magnetic agent has generally been presumed to be sufficient to remove the extracellular magnetic agent to an extent sufficient that the extracellular magnetic agent can be neglected during imaging intended to assess cell concentration. As disclosed herein, however, the extracellular magnetic agent remaining after such processing is generally not negligible, and release of magnetic contrast agent such as SPIO to extracellular space after cell death also causes substantial errors in quantitative analysis of cell concentration based on MR. Further, techniques disclosed herein provide more accurate quantification of the tagged cell concentration based on measurements of both R2 and R2* (or, equivalently, T2 and T2*) MR data from the subject in conjunction with calibration MR data acquired from phantoms containing various a priori known mixtures intracellular and extracellular magnetic agent.

The quantitative cell concentration assessment module 20 includes a T2 and T2* weighted image acquisition sub-module 22 that communicates with or is part of the MR control module 12 and causes the MR scanner 10 to acquire both T2-weighted and T2*-weighted images of the subject, or of a phantom containing intracellular magnetic agent, extracellular magnetic agent, or a mixture of intracellular and extracellular magnetic agent. In the illustrated embodiment, a series of T2-weighted images of the subject are acquired, a series of T2*-weighted images of the subject are acquired, and an R2 and R2* mapping sub-module 24 generates an R2 map of the subject and an R2* map based on the respective series of T2 and T2* weighted images.

With continuing reference to FIG. 1, in a calibration operation the sub-modules 22, 24 are employed to measure R2 and R2* for several phantoms containing different concentrations of intracellular magnetic agent with substantially no extracellular agent, and for several phantoms containing different concentrations of extracellular magnetic agent with substantially no intracellular agent. These measurements are used to generate calibration data 26 including: (i) a reference R2 relaxivity curve for intracellular magnetic agent; (ii) a reference R2* relaxivity curve for intracellular magnetic agent; (iii) a reference R2 relaxivity curve for extracellular magnetic agent; and (iv) a reference R2* relaxivity curve for extracellular magnetic agent.

For example, in an actually performed calibration, six phantoms were used to generate the calibration data 26. The six phantoms were six vials each of which was filled with 1 ml 1% agarose gel immersed in distilled water in a cylindrical glass tube. Three of the vials contained different concentrations of free SPIO (diluted from Feruomoxides). Three of the vials contained different concentrations of SPIO labeled C6 glioma cells. These six “pure” vials were used to generate calibration relaxation curves 26.

Each of the six phantom vials was measured using the sub-modules 22, 24. These illustrative MR scans were performed using a 3T clinical Achieva™ scanner (Achieva, Philips Healthcare, The Netherlands) with a 4 cm receive-only radio frequency coil (Philips Research Europe, Hamburg, Germany). MR images were acquired with a field-of-view (FOV) of 70 mm×70 mm, slice thickness=1 mm, data matrix=128×128, NEX=2. The R2* maps were acquired with a multiple gradient echo sequence: TR=900 ms, first TE/deltaTE=2.8 ms/1.8 ms, flip angle=30 degree, 25 echoes. The R2 maps were acquired with a turbo spin echo sequence with TR=1000 ms, first TE/delta TE=7 ms/7 ms, 20 echoes. These are merely illustrative scan parameters, and substantially any other scan configuration for acquiring R2 and R2* data is also suitable.

With continuing reference to FIG. 1 and with further reference to FIG. 2, the R2 and R2* values for each “pure” calibration phantom containing only intracellular SPIO or containing only extracellular SPIO were determined. The three R2 values obtained from the three phantom vials with SPIO labeled cells were fitted to generate the R2 relaxation curve for intracellular SPIO. The three R2* values obtained from the three phantom vials with SPIO labeled cells were fitted to generate the R2* relaxation curve for intracellular SPIO. The three R2 values obtained from the three phantom vials with free SPIO were fitted to generate the R2 relaxation curve for extracellular SPIO. The three R2* values obtained from the three phantom vials with free SPIO were fitted to generate the R2* relaxation curve for extracellular SPIO. In these fits, a linear relationship between R2 (or R2*) and the intracellular (or extracellular) concentration was assumed. The resulting relaxation curves are shown in FIG. 2.

FIG. 2 shows that the extracellular SPIO phantom vials have similar R2 and R2* relaxivities. Specifically, for extracellular SPIO the R2 reference relaxivity curve has a slope of 3.00 (ug/ml)⁻¹s⁻¹, while the R2* reference relaxivity curve has a slope of 3.70 (ug/ml)⁻¹s⁻¹. In sharp contrast, R2 and R2* relaxivities of intracellular SPIOs differ by large amounts. Specifically, the R2 reference relaxivity curve has a slope of 0.65 (ug/ml)⁻¹s⁻¹ while the R2* reference relaxivity curve has a slope of 8.24 (ug/ml)⁻¹s⁻¹.

As a result, it is recognized herein that for an unknown mixture of intracellular and extracellular magnetic tagging agent, if the R2 and R2* values are similar this indicates the sample is mostly free or extracellular magnetic tagging agent, whereas if the R2 value is much smaller than the R2* value this indicates the sample is mostly bound or intracellular magnetic tagging agent.

For a given mixture of intracellular magnetic agent and extracellular magnetic agent, the decay of the MR signal S(t) for a T2-weighted echo (such as a spin echo) is describable as a biexponential:

S(t)˜[intra]×exp(−t×R2([intra]))+[extra]×exp(−t×R2([extra])) (1)

where [intra] and [extra] are the concentrations of intracellular and extracellular magnetic tagging agent, respectively, and the symbol “˜” indicates a proportionality relationship. The constituent decay rates R2([intra]) and R2([extra]) are functions of the concentrations [intra] and [extra] as set forth in FIG. 2. In similar fashion, the decay of the MR signal S(t) for a T2*-weighted echo (such as a gradient echo) is describable as a biexponential:

S(t)˜[intra]×exp(−t×R2*([intra]))+[extra]×exp(−t×R2*([extra])) (2)

where again the decay rates R2*([intra]) and R2*([extra]) are functions of the concentrations [intra] and [extra] as set forth in FIG. 2. In Equations (1) and (2), the decay rates R2 and R2* can optionally be replaced by 1/T2 and 1/T2*, respectively, since R2=1/T2 and R2*=1/T2*.

In some embodiments it is contemplated to simultaneously fit Equations (1) and (2) to T2-weighted and T2*-weighted MR signals acquired from an unknown mixture of intracellular and extracellular magnetic agent, with the fitting parameters being the intracellular magnetic agent and extracellular magnetic agent concentrations [intra] and [extra] and a suitable amplitude scaling parameter or perameters, in order to quantitatively determine the concentrations [intra] and [extra]. However, such a fitting approach is computationally difficult, and may also be sensitive to noise in the data.

Accordingly, in an actually performed embodiment the estimation of the ratios of intracellular and extracellular SPIOs was determined using an approach employing the following operations. The R2* signal of the mixture was fitted with a monoexponential decay, thus giving an approximate R2* value. Then, assuming the mixture contained exclusively SPIO labeled cells, a first parameter R2intraSPIO of the vial was computed from the reference relaxivity curves of the intracellular SPIO based on the approximate R2*. In other words, the approximate R2* value was input to the lower-righthand plot of FIG. 2 to generate an intracellular iron concentration estimate which was then input to the lower-lefthand plot of FIG. 2 to generate parameter R2intraSPIO.

In similar fashion, assuming the mixture contained exclusively free SPIO, a second parameter R2extraSPIO of the vial was computed from the reference relaxivity curves of the extracellular SPIO based on the approximate R2*. In other words, the approximate R2* value was input to the upper-righthand plot of FIG. 2 to generate an extracellular iron concentration estimate which was then input to the upper-lefthand plot of FIG. 2 to generate parameter R2extraSPIO.

The R2 signal is then used. Specifically, the R2 signal of the mixture was then fitted with a biexponential decay model:

S(t)=a×exp(−t×R2intraSPIO)+b×exp(−t×R2extraSPIO) (3)

where here only a and b are unknown parameters. The ratio of intracellular and extracellular SPIOs was then estimated as the fitted ratio a/b. This information can then be used to reduce the number of fitted parameters in fitting Equation (1) and/or Equation (2). In an alternate approach, the approximate R2* obtained by monoexponential fitting of the T2*-weighted signal can be input to the lower-righthand plot of FIG. 2 to generate an intracellular iron concentration estimate which is the adjusted by the ratio a/b to provide an improved estimate of intracellular iron concentration.

With reference to FIG. 3, this latter approximate approach for approximating the solution to Equations (1) and (2) was tested using a set of seven phantoms. The phantoms were vials each filled with 1 ml 1% agarose gel immersed in distilled water in a cylindrical glass tube. The seven vials used for testing contained different mixtures of free SPIO and SPIO labeled cells in proportions adjusted to obtain different ratios of intracellular and extracellular SPIO concentrations. Details of these seven phantom vials containing mixtures of intracellular SPIO and extracellular SPIO are set forth in Table 1. As with the “pure” phantoms used in generating calibration data 26, these illustrative MR scans were performed using a 3T clinical Achieva™ scanner (Achieva, Philips Healthcare, The Netherlands) with a 4 cm receive-only radio frequency coil (Philips Research Europe, Hamburg, Germany). MR images were acquired with a field-of-view (FOV) of 70 mm×70 mm, slice thickness=1 mm, data matrix=128×128, NEX=2. The R2* maps were acquired with a multiple gradient echo sequence: TR=900 ms, first TE/deltaTE=2.8 ms/1.8 ms, flip angle=30 degree, 25 echoes. The R2 maps were acquired with a turbo spin echo sequence with TR=1000 ms, first TE/delta TE=7 ms/7 ms, 20 echoes. Again, these are merely illustrative scan parameters, and substantially any other scan configuration for acquiring R2 and R2* data is also suitable.

TABLE 1
Characteristics of the vials mixed with
SPIO labeled cells and free SPIOs.

| | vial 1 | vial 2 | vial 3 | vial 4 | vial 5 | vial 6 | vial 7 |
|---|---|---|---|---|---|---|---|
| SPIO labeled | 1.16 | 0.99 | 0.83 | 0.66 | 0.50 | 0.33 | 0.17 |
| cells (×10⁶) | | | | | | | |
| Free Iron (μg) | 0.75 | 1.50 | 2.25 | 3.00 | 3.75 | 4.50 | 5.25 |
| Intra SPIO/Extra SPIO | 4.62 | 1.98 | 1.10 | 0.66 | 0.40 | 0.22 | 0.09 |

The estimation of the ratios of intracellular and extracellular SPIOs in each of the seven different mixtures was performed with the following steps: (1) R2* of each mixture was fitted with a monoexponential decay; (2) assuming the mixture contained exclusively SPIO labeled cells, R2intraSPIO of the vial was computed from the reference relaxivity curves of the intracellular SPIO based on R2*; (3) similarly, R2extraSPIO of the vial was computed from the reference relaxivity curves of the extracellular SPIO assuming the mixture contained exclusively free SPIOs; (4) the R2 data of the mixture were then fitted with a biexponential decay model: S(t)=a×exp(−t×R2intraSPIO)+b×exp(−t×R2extraSPIO); and (5) the ratio of intracellular and extracellular SPIOs was estimated as a/b. As shown in FIG. 3, the estimated ratios (a/b) of the intracellular and extracellular SPIOs estimated from these reference relaxivities demonstrated a very good linear correlation with the theoretical values. The latter (that is, the theoretical values) were computed based on the magnetic agent load of the labeled cells (assumed to be approximately 3 pg/cell), which may subject to variations thereby cause the observed overestimation of the calculated ratios

These are merely illustrative approaches for quantitatively estimating the intracellular iron concentration based on both the T2-weighted image of the subject and the T2*-weighted image of the subject along with the calibration data 26. The quantitative estimation approaches disclosed herein entail approximate or exact simultaneous solution of Equations (1) and (2) based on received inputs including (1) measured R2 and R2* values for an unknown mixture and (2) the calibration data 26 for purely free magnetic agent and purely cell-bound magnetic agent such as that represented in FIG. 2.

With reference back to FIG. 1, the described processing can be performed at each spatial location, for example on a per-pixel or per-voxel basis, so that a quantitative cell concentration mapping sub-module 30 can generate a quantitative map of magnetically tagged cell concentration which can be displayed as an image by a cell concentration output sub-module 32 on a display 18d of the computer 18 or on another display device, printing device, or the like. In some embodiments, the ratio intracellular/extracellular concentration ratio a/b is assumed to be constant across the entire area of the R2 and R2* maps, or across an area of interest.

The display of the results can take various forms. In one approach, the spatially averaged concentration, maximum concentration anywhere in the image, or other aggregate magnetically tagged cell concentration is suitably output as a numerical display, graphical display (for example, a graphical bar whose length corresponds to the aggregate cell concentration), machine-generated speech representation, or other human-perceptible representation of a value indicative of quantitative assessment of magnetic agent-tagged cells in the subject. Additionally or alternatively, an image of the subject may be output, which is typically a magnetic resonance image although an image acquired by another modality is also contemplated, with this displayed image overlaid with a color-coded map of values indicative of quantitative assessment of magnetic agent-tagged cells in the subject. This latter display can be useful as a way to efficiently convey to the clinician, physician, or other medical expert the location or locations where the magnetically tagged cells are mostly highly concentrated and the location or locations where the magnetically tagged cells are sparsely concentrated or missing entirely.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A method for quantitative assessment of magnetic agent-tagged cells in a subject, the method comprising:
acquiring a series of T2-weighted images of the subject;
acquiring a series of T2*-weighted images of the subject; and
generating a value indicative of quantitative assessment of magnetic agent-tagged cells in the subject based on both the T2-weighted images of the subject and the T2*-weighted images of the subject.

2. The method as set forth in claim 1, further comprising:
outputting a numerical display, graphical display, machine-generated speech representation, or other human-perceptible representation of the value indicative of quantitative assessment of magnetic agent-tagged cells in the subject.

3. The method as set forth in claim 2, wherein the outputting comprises:
outputting an image of the subject; and
overlaying the image with a color-coded map of values indicative of quantitative assessment of magnetic agent-tagged cells in the subject.

4. The method as set forth in claim 1, further comprising:
administering cells to the subject wherein said cells are tagged with a superparamagnetic iron oxide (SPIO) agent.

5. The method as set forth in claim 1, wherein:
the acquiring a series of T2-weighted images of the subject comprises acquiring an R2 map of the subject;
the acquiring a series of T2*-weighted images of the subject comprises acquiring an R2* map of the subject; and
the generating operation comprises generating a value indicative of quantitative assessment of magnetic agent-tagged cells in the subject based on both the R2 map of the subject and the R2* map of the subject.

6. The method as set forth in claim 5, wherein the acquiring an R2 map of the subject comprises acquiring an image of the subject using a spin echo sequence.

7. The method as set forth in claim 5, wherein the acquiring an R2* map of the subject comprises acquiring an image of the subject using a gradient echo sequence.

8. The method as set forth in claim 5, wherein the generating a value indicative of quantitative assessment of magnetic agent-tagged cells in the subject based on both the R2 map of the subject and the R2* map of the subject is further based on calibration data comprising reference R2 and R2* relaxivity curves for intracellular magnetic agent and for extracellular magnetic agent.

9. The method as set forth in claim 5, wherein the generating a value indicative of quantitative assessment of magnetic agent-tagged cells in the subject based on both the R2 map of the subject and the R2* map of the subject is further based on calibration data comprising:
a relationship between R2 value and intracellular magnetic agent concentration for intracellular magnetic agent with substantially no extracellular magnetic agent,
a relationship between R2* value and intracellular magnetic agent concentration for intracellular magnetic agent with substantially no extracellular magnetic agent,
a relationship between R2 value and extracellular magnetic agent concentration for extracellular magnetic agent with substantially no intracellular magnetic agent, and
a relationship between R2* value and extracellular magnetic agent concentration for extracellular magnetic agent with substantially no intracellular magnetic agent.

10. The method as set forth in claim 8, further comprising:
generating said calibration data based on R2 and R2* measurements of a plurality of calibration phantoms having different concentrations of substantially purely intracellular magnetic agent and having different concentrations of substantially purely extracellular magnetic agent.

11. The method as set forth in claim 10, wherein the calibration phantoms include (i) at least three calibration phantoms having at least three different concentrations of substantially pure intracellular magnetic agent and (ii) at least three calibration phantoms having at least three different concentrations of substantially pure extracellular magnetic agent.

12. The method as set forth in claim 8, wherein the generating a value indicative of quantitative assessment of magnetic agent-tagged cells in the subject based on both the R2 map of the subject and the R2* map of the subject and further based on calibration data comprises:
estimating an intracellular magnetic agent concentration based on both the R2 map of the subject and the R2* map of the subject and further based on calibration data; and
converting the intracellular magnetic agent concentration to a cell concentration based on a magnetic agent load of the magnetically labeled cells.

13. The method as set forth in claim 1, wherein the generating comprises:
generating a value indicative of quantitative assessment of magnetic agent-tagged cells in the subject based on both the T2-weighted images of the subject and the T2*-weighted images of the subject, and further based on an predetermined relative similarity between R2 and R2* for extracellular magnetic agent and a predetermined relative dissimilarity between R2 and R2* for intracellular magnetic agent.

14. A magnetic resonance imaging system configured to perform a method as set forth in claim 1.

15. A digital storage medium storing instructions executable to cause a magnetic resonance imaging system to perform a method as set forth in claim 1.

16. A system for quantitative assessment of magnetic agent-tagged cells in a subject, the system comprising:
a magnetic resonance imaging system; and
a processor configured to cause the magnetic resonance imaging system to acquire both T2-weighted and T2*-weighted images of the subject and further configured to generate a value indicative of quantitative assessment of magnetic agent-tagged cells in the subject based on both the T2-weighted and T2*-weighted images.

17. The system as set forth in claim 16, wherein the processor is configured to generate a map indicative of quantitative assessment of a spatial distribution of magnetic agent-tagged cells in the subject based on both the T2-weighted and T2*-weighted images.

18. The method as set forth in claim 16, wherein the processor is configured to generate a value indicative of quantitative assessment of magnetic agent-tagged cells in the subject based on both the T2-weighted and T2*-weighted images and further based on predetermined relationships between (i) R2 and intracellular magnetic agent concentration, (ii) R2* and intracellular magnetic agent concentration, (iii) R2 and extracellular magnetic agent concentration, and (iv) R2* and extracellular magnetic agent concentration.

19. The method as set forth in claim 16, wherein the processor is configured to generate a value indicative of quantitative assessment of magnetic agent-tagged cells in the subject based on both the T2-weighted and T2*-weighted images and further based on quantitative information on (i) a relatively smaller divergence between R2 and R2* for extracellular magnetic agent and (ii) a relatively larger divergence between R2 and R2* for intracellular magnetic agent.

20. The method as set forth in claim 16, wherein the processor is configured to generate a value indicative of quantitative assessment of magnetic agent-tagged cells in the subject based on both the T2-weighted and T2*-weighted images by at least approximately solving the relationships:
S(t)˜[intra]×exp(−t×R2([intra]))+[extra]×exp(−t×R2([extra]))
and
S(t)˜[intra]×exp(−t×R2*([intra]))+[extra]×exp(−t×R2*([extra]))
where [intra] and [extra] are the concentrations of intracellular and extracellular magnetic tagging agent, respectively, R2([intra]) and R2*([intra]) are reference relaxivity curves obtained from substantially pure samples of intracellular magnetic agent, and R2([extra]) and R2*([extra]) are reference relaxivity curves obtained from substantially pure samples of extracellular magnetic agent.
